(19) United States
(12) Patent Application Publication
(10) Pub. No.: US 2016/0361514 A1
(43) Pub. Date: Dec. 15, 2016

(54) DISPENSING APPARATUS

(71) Applicant: CONSORT MEDICAL PLC, Hertfordshire (GB)

(72) Inventor: Richard WARBY, Cambridgeshire (GB)

(73) Assignee: CONSORT MEDICAL PLC, Hertfordshire (GB)

(21) Appl. No.: 14/739,538

(22) Filed: Jun. 15, 2015

(51) Int. Cl. A61M 16/20; A61M 11/00; A61M 16/10

(52) U.S. Cl. CPC A61M 16/20; A61M 16/104; A61M 16/109; A61M 11/00

(57) ABSTRACT

A dispensing apparatus (1) is provided comprising a fluid reservoir (4) and a valve (2). The fluid reservoir comprising a body which defines an internal volume which contains a fluid and the body comprising an open mouth.

The valve (2) is sealingly coupled to the body of the fluid reservoir (4) to close the open mouth and the valve comprises a gasket seal (18) which engages the body of the fluid reservoir.

The gasket seal (18) comprises an elastomer based on an ethylene-propylene-diene terpolymer, which terpolymer comprises from 45 to 55 wt. % ethylene, from 40 to 50 wt. % propylene, and ENB (ethylidenenorbornene) in an amount of 0.5 to 6 wt. %; and wherein the fluid comprises comprises a halogenated ether.

### FIELD

The present invention relates generally to a dispensing apparatus for use in dispensing inhalational anaesthetic for induction and maintenance of general anesthesia.

### BACKGROUND

An inhalational anaesthetic is a chemical compound possessing general anaesthetic properties that can be delivered via inhalation. They are typically administered by anaesthetists through, for example, an anaesthesia mask, laryngeal mask airway or tracheal tube connected to some type of anaesthetic vaporiser and an anaesthetic delivery system. Examples of volatile anaesthetic inhalational agents include nitrous oxide, xenon, ether and chloroform. Modern examples of are isoflurane, sevoflurane and desflurane, which compounds are all halogenated methyl ethyl ethers.

The delivery of the anaesthetic from a reservoir to a patient is usually accomplished through a vaporiser and a series of tubes and valves. An important valve is the valve which is sealing coupled to the anaesthetic reservoir. Such a valve will typically include a gasket seal which fills the space between two mating surfaces to prevent leakage from or into the joint.

Known rubber compounds for gasket seals for use in dispensing apparatus for dispensing an inhalational anaesthetic are based on the traditional technology of vulcanising a natural or synthetic or rubber polymer, for example nitrile rubber. The required material properties necessary for good seal performance include: chemical compatibility (swell), tensile strength, permanent compression set, stress relaxation and elastic modulus. These properties are often obtained in conjunction with a filler material such as, for example, carbon black.

Products to be dispensed are commonly provided in solution or suspension in an alcohol base, this being particularly common in the dispensing of medicinal compounds for inhalation therapy.

It has been found that certain conventional gasket seals may discolour halogenated methyl ethyl ethers. Such discolouration may be unattractive to the patient. Moreover, it may mask, or be mistaken for, leakage and deterioration of the anaesthetic.

The present invention aims to address or mitigate problems associated with the prior art.

### SUMMARY OF THE DISCLOSURE

The present invention provides a dispensing apparatus comprising a fluid reservoir and a valve;

the fluid reservoir comprising a body which defines an internal volume which contains a fluid and the body comprising an open mouth;

the valve being sealingly coupled to the body of the fluid reservoir to close the open mouth;

wherein the valve comprises a gasket seal which engages the body of the fluid reservoir;

wherein the gasket seal comprises an elastomer based on an ethylene-propylene-diene terpolymer, which terpolymer comprises from 45 to 55 wt. % ethylene, from 40 to 50 wt. % propylene, and ENB (ethylidenenorbornene) in an amount of 0.5 to 6 wt. %; and

wherein the fluid comprises an anaesthetic liquid comprising a halogenated ether.

The term gasket seal as used herein is intended to encompass any sealing member or portion thereof present in a dispensing apparatus, including, but not limited to, gaskets and seals whether static or dynamic.

Preferably, the terpolymer comprises from 48 to 52 wt. % ethylene, from 43 to 47 wt. % propylene and from 2 to 5 wt. % ethylidene-norbornene (ENB). Still more preferably the terpolymer comprises approximately 50 wt. % ethylene, approximately 45 wt. % propylene and approximately 5 wt. % ENB. The ethylene content may be determined by ASTM D3900. The propylene content may be determined by ASTM D3900. The ENB content may be determined by ASTM D6047.

The terpolymer preferably has a residual transition metal content of <20 ppm, more preferably <10 ppm.

The terpolymer is preferably manufactured using a constrained geometry catalyst system, preferably a metallocene constrained geometry catalyst system. For example, one based on titanium monocyclopentadienyl, preferably with a silane group incorporated therein.

The terpolymer is preferably substantially free of any catalyst residuals. The terpolymer typically has a Mooney Viscosity (ML 1+4, 125° C.) of from 10 to 60, preferably from 15 to 40, more preferably from 15 to 30, still more preferably from 20 to 30. The Mooney Viscosity may be determined by ASTM D1646.

The terpolymer typically has a medium molecular weight distribution.

The terpolymer typically has a density of from 0.84 to 0.88, more typically approximately 0.86.

The terpolymer typically has a density of from 0.84 to 0.90 g/cm³, more typically from 0.85 to 0.87 g/cm³, more typically approximately 0.86 g/cm³. The density may be determined by ASTM D297.

The terpolymer typically has an ash content of <0.1 wt. %, and a total volatiles content of <0.4 wt. %.

The material for the gasket seal preferably further includes a filler, more preferably a mineral filler.

Mineral fillers are preferable to carbon black in order to minimise the formation of polynuclear aromatic hydrocarbon compounds. Suitable examples include any of magnesium silicate, aluminium silicate, silica, titanium oxide, zinc oxide, calcium carbonate, magnesium oxide magnesium carbonate, magnesium aluminium silicate, aluminium hydroxide, talc, kaolin and clay, including combinations of two or more thereof. Preferably, the filler is or comprises one or more of magnesium silicate, talc, calcified clay, and/or kaolin. The most preferred filler for use with the material for the gasket seal comprises one or both of silica and/or talc.

The gasket seal may comprise, for example, 40-50 wt. % of the filler (for example silica and talc) and 45-55 wt. % of the terpolymer.

The material for the gasket seal will typically further include a process aid, such as, for example, stearic acid. This may be provided in the gasket seal in an amount of up to 0.7 wt. %.

The material for the gasket seal will typically further include a curing or cross-linking agent. For example, the material may further include a peroxide curing agent, sulphur or a sulphur-containing compound. A peroxide curing agent such as, for example, dialkyl peroxide is, however, preferable to other curing agents such as sulphur since its use minimises the formation of extractives (e.g. 2-mercaptobenzothiazole, N-nitrosamines, mercaptobenzothiazole disulphide) resulting from contact between the material and alcohol in use. The curing or cross-linking agent (for example a dialkyl peroxide) may be provided in the gasket seal in an amount of up to 3 wt. %.

The material for the gasket seal may further include an antioxidant, such as, for example, octylated diphenylamine. This may be provided in the gasket seal in an amount of up to 0.7 wt. %.

The material for the gasket seal may further include one or more of a reinforcement agent, a plasticizer, a binder, a stabilizer, a lubricant and a pigment. One or more of these may be provided in the gasket seal in an amount of up to 1 wt. %.

The gasket seal may have been subjected to an alcohol extraction, for example an ethanol extraction, to reduce or eliminate extractives and/or leachables.

It will be appreciated that the seal may be provided as a separate component or may be formed integrally with the valve.

The seal of the present invention may be manufactured by any of the processes conventional in the art. For example, the seal may be manufactured by compression moulding, injection moulding or extrusion.

A process for the preparation of the gasket seal may comprise the following steps:

(i) forming a composition comprising a mixture of an ethylene-propylene-diene terpolymer, which terpolymer comprises from 45 to 55 wt. % ethylene, from 40 to 50 wt. % propylene and 0.5 to 6 wt. % diene (for example, ethylidenenorbornene), a cross-linking agent for the terpolymer, a mineral filler and optionally a process aid; (ii) initiating a cross-linking reaction in the mixture to form a cross-linked elastomeric composition; and (iii) either before or after (ii) forming the composition into a gasket seal. The step of forming the composition into a seal will typically involve one or more forming techniques selected from compression moulding, injection moulding and extrusion. The terpolymer is preferably manufactured using a constrained geometry catalyst system.

An alcohol extraction (for example an ethanol extraction) of the gasket seal is preferably performed after the seal has been manufactured and before the valve is assembled. This step reduces or eliminates extractives and/or leachables.

Nordel IP 4520 (trade name) is a preferred EPDM terpolymer for use in the gasket seal material according to the present invention.

Nordel IP 4520 exhibits limited chemical interaction with drug and other component parts.

Nordel IP 4520 is polymerized by a constrained geometry catalyst. This delivers precise control of, for example, Mooney viscosity, ethylene content and ENB content for highly predictable rheology and cure rates that can enhance valve performance in a dispensing apparatus. The production process also ensures precise control of the molecular weight distribution.

Because of the high efficiency of the constrained geometry catalyst, Nordel IP 4520 is a relatively clean and uniform EPDM elastomer, with substantially no catalyst residuals and reduced metal content compared with conventional Ziegler-Natta catalysed EPDM elastomers.

Surprisingly it has been found that the use of a gasket seal as herein described reduces or eliminates discolouration of anaesthetic formulations, particularly those comprising halogenated ethers. Examples of anaesthetic formulations are those comprising or consisting of desflurane (1,2,2,2-tetrafluoroethyl difluoromethyl ether), sevoflurane (1,1,1,3,3,3-hexafluoro-2-(fluoromethoxy)propane (also known as fluoromethyl hexafluoroisopropyl ether)) and isoflurane (2-chloro-2-(difluoromethoxy)-1,1,1-trifluoro-ethane). The gasket seal finds particular application where the anaesthetic formulation comprises a halogenated methyl ethyl ether, for example a fluorinated methyl ethyl ether.

There is also provided an anaesthetic machine that comprises a dispensing apparatus as herein described. Such machines are typically fitted with an anaesthetic vaporiser unit that heats the liquid anaesthetic formulation to a constant temperature. This enables the agent to be available at a constant vapor pressure, negating the effects fluctuating ambient temperatures would otherwise have on its concentration imparted into the fresh gas flow of the anaesthetic machine.

The present invention will now be further described with reference to the following non-limiting drawings.

### BRIEF DESCRIPTION OF THE DRAWINGS

Embodiments of the present disclosure will now be described, by way of example only, with reference to the accompanying drawings, in which:

FIG. 1 is a side view of a dispensing apparatus according to the present disclosure;

FIG. 2 is a cross-sectional view of a valve of the dispensing apparatus of FIG. 1;

FIG. 3 is a sectioned perspective view of the valve of FIG. 2;

FIG. 4 is an exploded perspective view of the valve of FIG. 2; and

FIG. 5 is a schematic view showing coupling of the dispensing apparatus to an external device.

### DETAILED DESCRIPTION

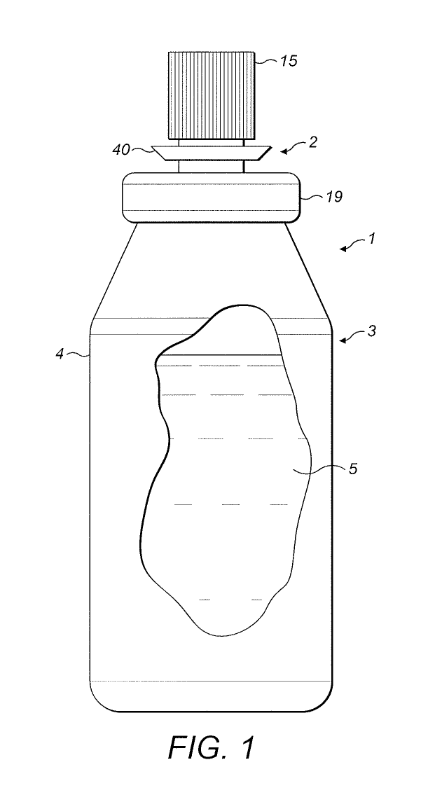

As shown in FIG. 1, a dispensing apparatus 1 according to the present disclosure comprises a fluid reservoir 4 and a valve 2. The fluid reservoir 4, which may for example be in the form of a bottle, has a body which defines an internal volume which can contain a fluid 5 intended to be dispensed in use via the valve 2. The fluid reservoir 4 may be formed from a suitable material which is non-reactive with the fluid 5. Examples include glass, metals (e.g. stainless steel or aluminium) and engineering plastics.

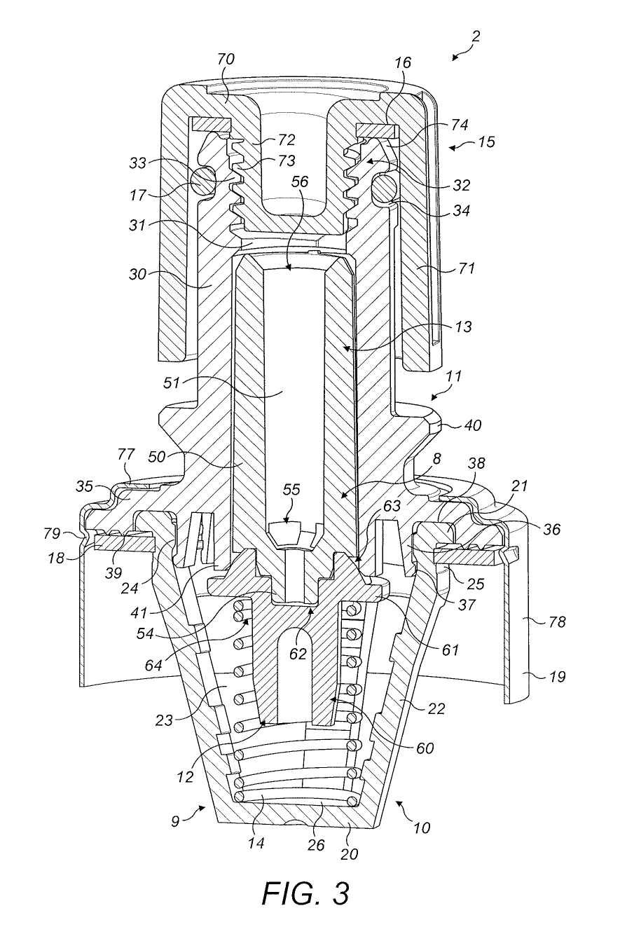

In the following description, relative positional terms such as “upper”, “lower”, “inner” and “outer” are to be understood to refer to the dispensing apparatus 1 or valve 2 in the orientation as shown in FIGS. 1 and 3—that is with the dispensing outlet of the valve 2 uppermost. However, it will be appreciated that in use the dispensing apparatus 1 may adopt different orientations and the relative positional terms used herein are used for ease of reference only and will be understood not to require any absolute orientation of the dispensing apparatus 1 or valve 2.

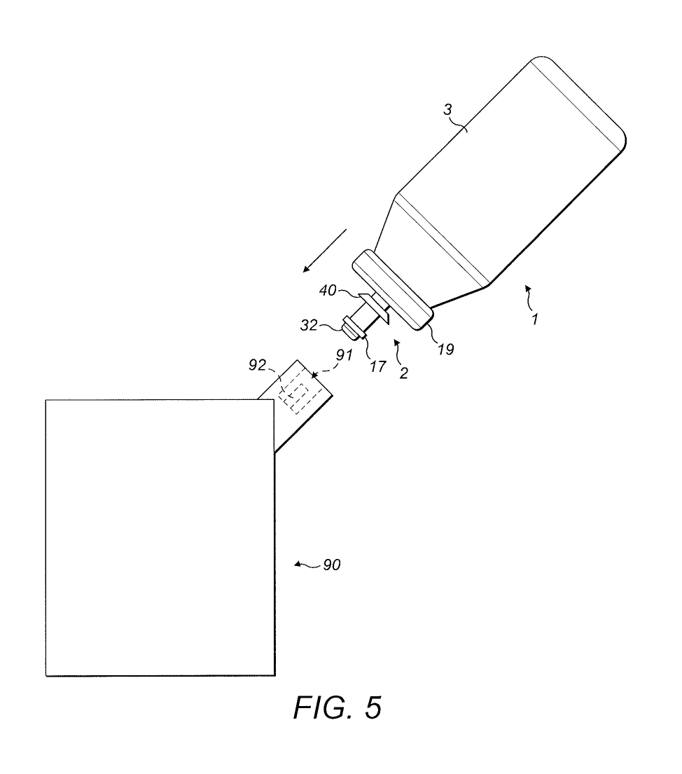

The fluid 5 may be a liquid at standard temperature and pressure. In one example the fluid 5 may be an anaesthetic agent intended to be administered to humans or animals by inhalation. As shown schematically in FIG. 5, the anaesthetic agent may be administered by means of an external device 90 in the form of an anaesthetic machine which comprises a vaporiser unit. The dispensing apparatus 1 may be coupled to the external device 90 in order to transfer the fluid 5 to the vaporiser unit as will be described below. The fluid 5 may, for example, comprise or consist of desflurane, sevoflurane, isoflurane or a combination of two or more thereof.

The body of the fluid reservoir 4 comprises an open mouth at one end which is closed off by the valve 2 to prevent exit of the fluid 5 other than via the valve 2.

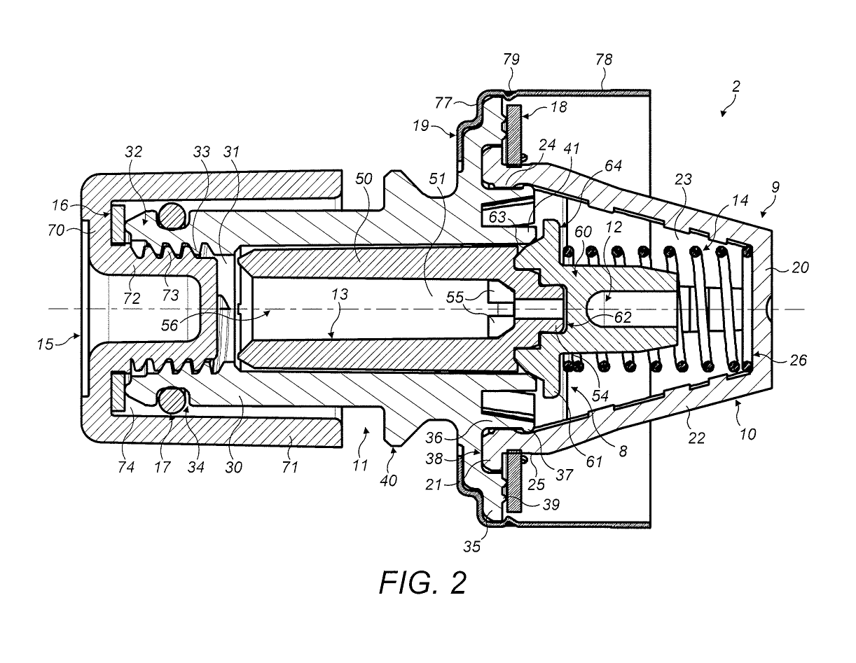

FIGS. 2 to 4 show details of the valve 2. The valve 2 comprises a valve body 9, a core 8, a spring 14, a cap 15 and a ferrule 19. In addition, a plurality of seals are provided: a gasket seal 18 is provided to seal the valve body 9 to the fluid reservoir 4; a cap seal 16 is provided to seal the cap 15 to the valve body 9; and an O-ring seal 17 is provided to seal the valve body 9 to an external device 90 when the dispensing apparatus 1 is used to discharge the fluid 5 from the fluid reservoir 4 as will be described further below.

In basic operation, the valve 2 acts as a ‘poppet’ valve wherein the valve body 9 remains static relative to the fluid reservoir 4 and the core 8 is axially moveable within the valve body 9 against the bias of the spring 14 to open an exit path for fluid 5 to leave the fluid reservoir 4 via the valve 2.

As shown in detail in FIGS. 2 and 3, the valve body 9 comprises a lower valve body 10 and an upper valve body 11 which are coupled together surrounding the core 8. The lower valve body 10 has a generally frusto-conical form with a base 20 which is relatively small in diameter and four struts 22 which extend upwards and taper outwards from the base 20 to join with an upper flange 21 which extends circumferentially about the valve body 9. Thereby, four windows 23 are formed in the lower valve body 10, each window 23 being bounded on either side by one of the struts 22 and at a lower end by the base 20 and at an upper end by the upper flange 21. An upper end of the lower valve body 10 at or near the upper flange 21 is provided with a circumferential ridge 24 on an inner surface and with a circumferential depression 25 on an outer surface. An inner surface of the base 20 forms a lower spring seat 26 against which one end of the spring 14 abuts after assembly as shown in FIG. 3. The lower valve body 10 may be formed as a single, unitary molding. The lower valve body 10 may preferably be formed from an engineering plastic, e.g. polyamide.

The upper valve body 11 comprises a neck 30, which may have the form of an elongate body, wherein the neck 30 defines a central bore 31. An upper end 32 of the neck 30 defines an outlet of the central bore 31 which defines the dispensing outlet of the dispensing apparatus 1. The upper end 32 of the upper valve body 11 also comprises an O-ring seat 34 on its outer surface in the form of a circumferential recess which receives the O-ring seal 17 therein on assembly. The upper end 32 is further provided with an internal screw thread 33 for coupling to the cap 15. A flange 35 is provided at a lower end of the upper valve body 11. The flange 35 is shaped to provide an annular recess 38 on a lower surface of the flange 35 which is sized and shaped to receive the upper flange 21 of the lower valve body 10. A portion of the flange 35 lying radially outside of the annular recess 38 forms a lower sealing surface of the flange 35 and may be provided with one or two circumferential raised ridges 39. The lower valve body 10 is also provided with a plurality of attachment legs 36 which extend downwardly from a lower end of the flange 35. Each attachment leg 36 is provided at its distal end with a detent 37 which faces outwards. A lower end of the neck 30 comprises an inner rim 41 which demarcates a lower inlet to the central bore 31. The upper valve body 11 is further provided on its exterior with a connection flange 40 which is located mid-way along the length of the upper valve body 11. The connection flange 40 comprises a flat upper face configured to engage a mating surface of the external device 90 during filling. The upper valve body 11 may be formed as a single, unitary molding. The upper valve body 11 may preferably be formed from an engineering plastic, e.g. polyamide.

The lower valve body 10 and upper valve body 11 are assembled together to form the valve body 9 as shown in FIG. 3 by being clipped together. As can be seen in FIG. 3, clipping together results in the attachment legs 36 being slindingly received within the upper flange 21 of the lower valve body 10. The attachment legs 36 flex during coupling to facilitate the passing of the detents 37 over and past the circumferential ridge 24. Once past the circumferential ridge 24 the attachment legs 36 flex back such that the detents 37 retainingly engage below the circumferential ridge 24. In this state, the upper flange 21 has been received within the annular recess 38 and the core 8 is captive within the valve body 9.

The core 8 comprises a lower core 12 and an upper core 13 which are coupled together by a suitable means, for example an interference fit. The lower core 12 comprises a body 60 which has a lower cylindrical portion provided with a flange 61 at its upper end. The upper end of the body 60 defines a socket 62. A domed sealing surface 63 is disposed annularly around the socket 62. A lower surface of the flange 61 forms an upper spring seat 64 for receiving one end of the spring 14 as shown in FIG. 3.

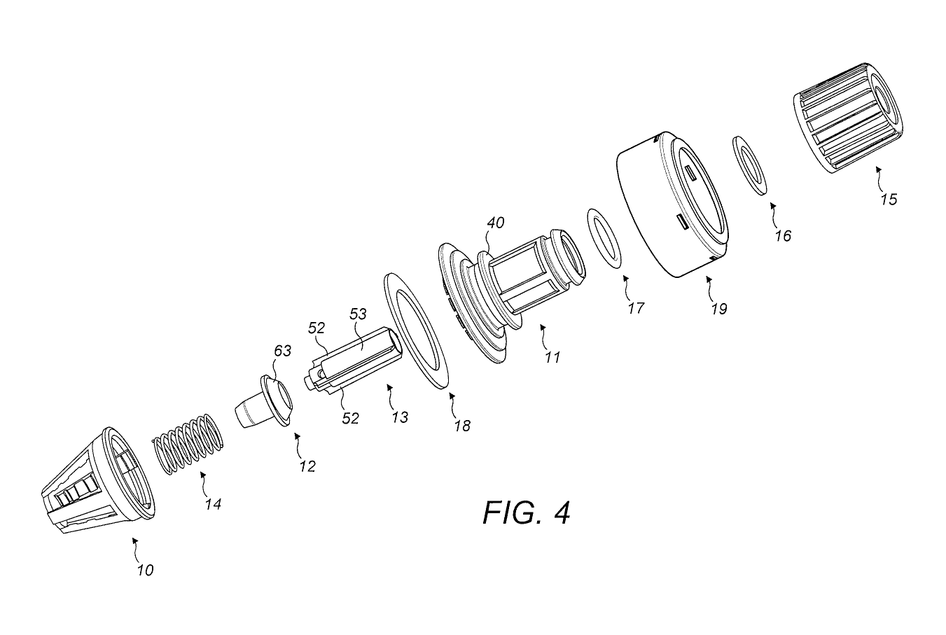

The upper core 13 comprises an elongate body 50 which defines an internal bore 51. An external surface of the elongate body 50 is provided with a plurality of axially extending ribs 52 defining external channels 53 there between as best seen in FIG. 4. A lower end of the elongate body 50 forms a plug 54 which can be matingly received in the socket 62 of the lower core 12. An upper end of the elongate body 50 defines an opening 56. Fluid communication into the internal bore 51 is permitted by the provision of four apertures 55 circumferentially arranged around the elongate body 50 at a lower end thereof just above the location of the plug 54 as shown in FIGS. 3 and 4.

The core 8 is slidingly movable within the valve body 9 between a closed configuration as shown in FIGS. 2 and 3—wherein the lower core 12 sealingly engages the inner rim 41 of the upper valve body 11—and an open configuration where the lower core 12 is disengaged from the inner rim 41 as will be described in more detail below.

The lower core 12 and upper core 13 may each be formed as a single, unitary molding. The upper core 13 may preferably be formed from an engineering plastic, e.g. polyamide, while the lower core 12 is preferably formed from a softer plastic, for example LDPE.

The spring 14 may be a helical spring formed from stainless steel.

The ferrule 19 comprises an upper portion 77 which overlies the flange 35 of the upper valve body 11 and a dependent skirt 78 which extends downwards beyond the location of the gasket seal 18. The ferrule 19 may be formed from aluminium. The ferrule 19, prior to crimping, may be provided with nibs 79 which aid retention of the ferrule 19 to the flange 35 of the neck 30.

The cap 15 may comprise a cup shaped member having an upper end 70 and a dependent side wall 71. The upper end 70 is provided with an inner extension 72 having provided thereon an outward facing screw thread 73. The dependent side wall 71 and inner extension 72 define there between an annular recess 74 in which is received the cap seal 16 as an interference fit. As shown in FIG. 3, the outward facing screw thread 73 of the inner extension 72 is engageable with the internal screw thread 33 of the upper valve body 11 to allow the cap 15 to be screwed onto and off the upper valve body 11 in use. The cap 15 may be formed as a single, unitary molding. The cap 15 may preferably be formed from an engineering plastic, e.g. polyamide.

The cap seal 16 may be in the form of an annular seal which is locatable around the inner extension 72 as shown in FIG. 2. The cap seal 16 may comprise an EPDM material. The cap seal 16 may comprise an EPDM material and optional processing aids and optional filler materials as herein described in relation to the gasket seal 18.

The O-ring seal 17 is sized to be located in the O-ring seat 34 of the upper valve body 11 and form an interference fit therewith. The O-ring seal 17 may comprise an EPDM material.

The gasket seal 18 may be in the form of an annular seal which is locatable around the lower valve body 10 as shown in FIG. 3. An upper face of the gasket seal 18 is engageable against the lower face of the flange 35 of the upper valve body 11 and/or the lower face of the upper flange 21 of the lower valve body 10. Where circumferential raised ridges 39 are provided on the lower face of the flange 35, these may bite into the upper surface of the gasket seal 18 improving the sealing contact there between. An inner face of the gasket seal 18 may form an interference fit with the outer surface of the lower valve body 10 and in particular may be engaged into the circumferential depression 25 formed in the lower valve body 10. This interengagement between the gasket seal 18 and the circumferential depression 25 may help to retain the gasket seal 18 in place on assembly of the valve 2 before the valve 2 is coupled to the fluid reservoir 4. The gasket seal 18 comprises an elastomer as herein described, namely one based on an ethylene-propylene-diene terpolymer, which terpolymer comprises from 45 to 55 wt. % ethylene, from 40 to 50 wt. % propylene, and ENB (ethylidenenorbornene) in an amount of 0.5 to 6 wt. %, and optional processing aids and optional filler materials.

To assembly the dispensing apparatus 1, the fluid reservoir 4 is first filled with the fluid 5 and the valve 2 is then coupled to the fluid reservoir 4 and retained thereon by use of the ferrule 19. The dependent skirt 78 of the ferrule 19 is crimped over a neck of the fluid reservoir 4 to provide a mechanical connection between the valve 2 and the fluid reservoir 4. During the crimping process an upper rim of the fluid reservoir 4 is engaged sealingly with the gasket seal 18.

During storage of the dispensing apparatus 1, the cap 15 is left screwed onto the upper valve body 11 with the cap seal 16 sealingly engaging the upper end 32 of the upper valve body 11. The compressive sealing interface between the gasket seal 18 and the upper rim of the fluid reservoir 4 prevents leakage of fluid 5 from between the fluid reservoir 4 and the valve 2. In the storage configuration, the core 8 rests in the closed configuration as shown in FIG. 3 with the spring 14 biasing the core 8 upwards so that the domed sealing surface 63 of the lower core 12 sealingly contacts the inner rim 41 of the upper valve body 11. Because of this seal, fluid 5 cannot pass into the internal bore 51 or external channels 53. Thus, in the closed configuration of the valve 2 the only seal exposed to the bulk volume of the fluid 5 contained in the fluid reservoir 4 is the gasket seal 18.

In use of the dispensing apparatus 1, a user will first remove the cap 15 by unscrewing it, and will then engage the upper end 32 of the upper valve body 11 with the external device 90 which is to be filled with the fluid 5. This arrangement is shown schematically in FIG. 5. The external device 90 comprises a socket 91 shaped and configured to receive the upper end 32 of the upper valve body 11. The socket 91 is further provided with one or more actuating prongs 92 which contact and engage the upper end of the elongate body 50 of the core 8. As the dispensing apparatus 1 is pushed into the socket 91 the actuating prongs 92 depress the core 8 relative to the static valve body 9 which moves both the upper core 13 and the lower core 12 downwards into the open configuration of the valve 2 so as to unseal the domed sealing surface 63 of the lower core 12 from the inner rim 41. In this open configuration, a fluid communication path between the fluid reservoir 4 and the open upper end 32 of the upper valve body 11 is achieved. The fluid 5 may then be drained from the fluid reservoir 4 by inverting the dispensing apparatus 1 (if not already in this orientation) so that the valve 2 is lowermost such that the fluid 5 drains from the fluid reservoir 4 through the windows 23 of the lower valve body 10 and into the external channels 53 formed between the upper core 13 and the upper valve body 11. The fluid 5 is able to pass along these external channels 53 and in addition may enter through the apertures 55 into the internal bore 51 and pass there through. The provision of multiple flow conduits within the valve 2, for example the internal bore 51 and the external channels 53, helps to prevent pulsing or “glugging” in the outflow of fluid which can occur where fluid exiting the valve 2 blocks off incoming air which is attempting to enter the fluid reservoir 4 to equalise pressures.

The flat face of the connection flange 40 may abut against the socket 91 of the external device 90 to limit the degree of insertion of the valve 2 into the external device 90. In addition the angled face of the connection flange 40 may be used in conjunction with a retention/locking mechanism of the external device 90 to ensure a positive connection between the valve 2 and the external device 90 by means of the retention/locking mechanism ‘pulling’ on the connection flange 40 to urge the upper end 32 into the socket 91.

After draining of the fluid 5, the dispensing apparatus 1 can be detached from the external device 90 and if desired the cap 15 re-screwed onto the upper valve body 11 if the contents are not exhausted.

In a modified embodiment of the present disclosure, the lower core 12 may be modified to replace the domed sealing surface 63 with a separately formed core seal. The core seal would be mounted to the lower core 12 and be biased into sealing contact with the inner rim 41 in the closed configuration of the valve 2. The core seal may be formed from the same material(s) as the gasket seal 18 as described above.

1. Dispensing apparatus comprising a fluid reservoir and a valve;
the fluid reservoir comprising a body which defines an internal volume which contains a fluid and the body comprising an open mouth;
the valve being sealingly coupled to the body of the fluid reservoir to close the open mouth;
wherein the valve comprises a gasket seal which engages the body of the fluid reservoir;
wherein the gasket seal comprises an elastomer based on an ethylene-propylene-diene terpolymer, which terpolymer comprises from 45 to 55 wt. % ethylene, from 40 to 50 wt. % propylene, and ENB (ethylidenenorbornene) in an amount of 0.5 to 6 wt. %; and
wherein the fluid comprises comprises a halogenated ether.

2. Dispensing apparatus as claimed in claim 1, wherein the terpolymer comprises from 48 to 52 wt. % ethylene, from 43 to 47 wt. % propylene, and from 2 to 5 wt. % ENB.

3. Dispensing apparatus as claimed in claim 2, wherein the terpolymer comprises approximately 55 wt. % ethylene, approximately 40 wt. % propylene, and approximately 5 wt. % ENB.

4. Dispensing apparatus as claimed in claim 1, wherein the terpolymer has a residual transition metal content of <20 ppm.

5. Dispensing apparatus as claimed in claim 1, wherein the terpolymer is manufactured using a constrained geometry catalyst system.

6. Dispensing apparatus as claimed in claim 1, wherein the terpolymer has substantially no catalyst residuals.

7. Dispensing apparatus as claimed in claim 1, wherein the terpolymer has a Mooney Viscosity (ML 1+4, 125 C) of from 10 to 60.

8. Dispensing apparatus as claimed in claim 1, wherein the gasket seal further comprises a mineral filler.

9. Dispensing apparatus as claimed in claim 8, wherein the mineral filler is selected from one or more of magnesium silicate, aluminium silicate, silica, titanium oxide, zinc oxide, calcium carbonate, magnesium oxide magnesium carbonate, magnesium aluminium silicate, aluminium hydroxide, talc, kaolin and clay.

10. Dispensing apparatus as claimed in claim in claim 1, wherein the gasket seal further comprises a process aid.

11. Dispensing apparatus as claimed in claim 1, wherein the gasket seal further comprises a peroxide curing agent and/or a sulphur curing agent.

12. Dispensing apparatus as claimed in claim 11, wherein the curing agent is a dialkyl peroxide.

13. Dispensing apparatus as claimed in claim 1, wherein the gasket seal further comprises one or more of a reinforcement agent, a plasticizer, a binder, a stabilizer, a retarder, a bonding agents, an antioxidant, a lubricant, a pigment, a wax, a resin, an antiozonants, a primary accelerator, a secondary accelerator or an activator.

14. Dispensing apparatus as claimed in claim 1, wherein the gasket seal has been subjected to an alcohol extraction to reduce or eliminate extractives and/or leachables.

15. Dispensing apparatus as claimed in claim 1, wherein the halogenated ether is a halogenated methyl ethyl ether.

16. Dispensing apparatus as claimed in claim 1, wherein the halogenated ether is selected from the group consisting of 1,2,2,2-tetrafluoroethyl difluoromethyl ether, 1,1,1,3,3,3-hexafluoro-2-(fluoromethoxy)propane and 2-chloro-2-(difluoromethoxy)-1,1,1-trifluoro-ethane.

17. An anaesthetic machine comprising a dispensing apparatus as defined in claim 1.

18. An anaesthetic machine as claimed in claim 17, further comprising an anaesthetic vaporiser unit.
